(19) United States
(12) Patent Application Publication
(10) Pub. No.: US 2018/0338556 A1
(43) Pub. Date: Nov. 29, 2018

(54) Cool Strip

(71) Applicant: Charla Cothran, Memphis, TN (US)

(72) Inventor: Charla Cothran, Memphis, TN (US)

(21) Appl. No.: 15/992,116

(22) Filed: May 29, 2018

Related U.S. Application Data
(60) Provisional application No. 62/511,874, filed on May 26, 2017.

(51) Int. Cl. A42B 1/00; A61B 5/01; A61B 5/00; A61F 7/10

(52) U.S. Cl. CPC A42B 1/008; A61B 5/01; A61F 2007/0007; A61B 5/6831; A61F 7/10; A61B 5/6814

(57) ABSTRACT

A cooling device is disclosed herein. The disclosed cooling device comprises a sensor or thermometer that indicates body temperature and contains an activatable cooler. In some versions, the disclosure discusses an activatable cooler comprising water and an endothermic salt. The device is designed to promote safety and comfort of the user-wearer.

### CROSS REFERENCE TO RELATED APPLICATION

The present application is related to and claims priority to U.S. Provisional Patent Application No. 62/511,874 filed May 26, 2017, which is incorporated by reference herein in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### 1. FIELD OF THE INVENTION

The present invention relates generally to the field of wearable cooling devices and more specifically relates to instant cold packs.

### 2. DESCRIPTION OF RELATED ART

Heat-related illness is a spectrum of disorders due to environmental exposure to heat. It includes minor conditions such as heat cramps, heat syncope, and heat exhaustion as well as the more severe condition known as heat stroke. Preventive measures include drinking plenty of cool liquids and avoiding excessive heat and humidity. Treatment requires rapid physical cooling of the body.

U.S. Pat. No. 8,061,891 to Lin et al. relates to an adhesive thermometer. The described adhesive thermometer includes an adhesive thermometer, comprising: a sense unit, a display unit and an attachment unit, where the display unit and the sense unit are combined into a rectangular parallelepiped, and the display unit is located at the top while the sense unit at the bottom. The attachment unit is formed by an adhesive pad and the rectangular parallelepiped, which makes the sense unit to be disposed for exposing downward. The bottom of the adhesive pad is provided with gluey substance and a protective pad is stuck to the gluey substance for temporary attachment. When using, simply tear down the protective pad and stick the thermometer to both ends of a measured location by means of the exposed gluey substance and adhere the sense unit to the measured location to sense the temperature, followed by exhibiting the sensed temperature at the display unit in terms of different message types: numeral, color, sound or odor.

Such thermometers can send skin temperature of a patient but are unable to counter those temperatures when the temperatures reach dangerous levels. A sensing device combined with the treating device is needed.

### BRIEF SUMMARY OF THE INVENTION

In view of the foregoing disadvantages inherent in the known wearable cooling device art, the present disclosure provides a novel cool strip or cooling device. The general purpose of the present disclosure, which will be described subsequently in greater detail, is to provide a cooling device that indicates body temperature and contains an activatable cooler.

A cooling device is disclosed herein. The cooling device includes a pouch; an activatable cooler disposed in the pouch; substrate disposed in the pouch; a thermometer affixed to the pouch; and a connector on an outer surface of the pouch wherein the connector secures the pouch to a body region.

A method of using cooling devices is also disclosed herein. The method of using cooling devices may comprise the steps of: providing a cooling device including a pouch; an activatable cooler disposed in the pouch; substrate disposed in the pouch; a thermometer affixed to the pouch; and a connector on an outer surface of the pouch wherein the connector secures the pouch to a body region. Other steps include securing the cooling device to a body region and activating a cooler.

For purposes of summarizing the invention, certain aspects, advantages, and novel features of the invention have been described herein. It is to be understood that not necessarily all such advantages may be achieved in accordance with any one embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a cool strip, constructed and operative according to the teachings of the present disclosure.

FIG. 1 is a perspective view of the cooling device during an ‘in-use’ condition, according to an embodiment of the disclosure.

FIG. 2 is a perspective view of the cooling device of FIG. 1, according to an embodiment of the present disclosure.

FIG. 3A is a front view of the cooling device of FIG. 1, according to an embodiment of the present disclosure.

FIG. 3B is a left side view of a version of the cooling device of FIG. 1;

FIG. 3C is a rear view of a version of the cooling device of FIG. 1.

FIG. 4 is a flow diagram illustrating a method of use for the cooling device, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to a wearable cooling device and more particularly to a cool strip as used to improve the instant cold packs.

Generally, the present invention provides users with a small gel cooling patch or strip designed to reduce body temperature when applied to the neck, armpits, or groin area. It measures the wearer's temperature immediately when applied, notifying the individual of their body temperature. It activates a cooling mechanism when squeezed, allowing the user's temperature to be reduced while waiting for help to arrive. This eliminates the worry of the body reaching extremely dangerous temperatures when the individual is suffering from heat exhaustion, fever, fainting, or seizures. The present invention functions as a cooling tool for those working outdoors and in other hot environments.

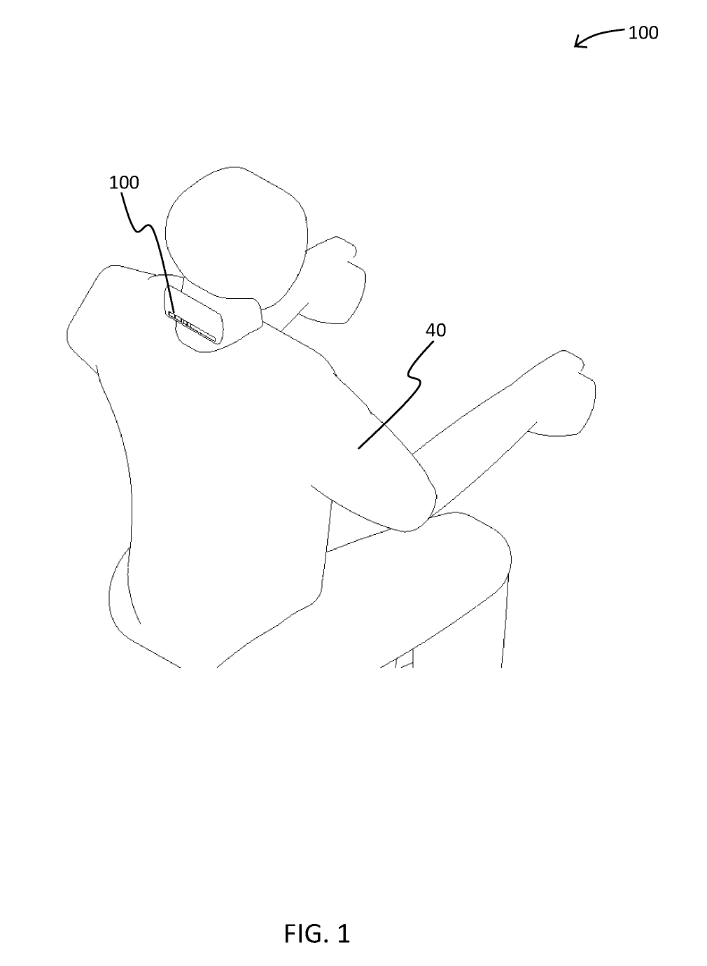

Referring now more specifically to the drawings by numerals of reference, there is shown in FIGS. 1-4, various views of cooling device 100. FIG. 1 shows cooling device 100 during an ‘in-use’ condition by user 40, according to an embodiment of the present disclosure.

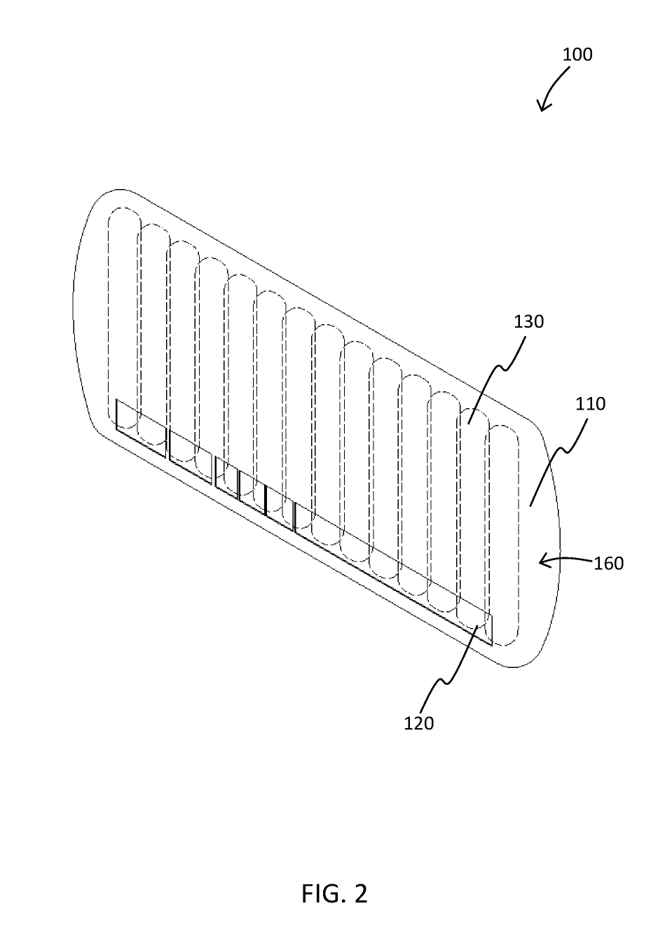

FIG. 2 shows a perspective view of cooling device 100 of FIG. 1, according to an embodiment of the present disclosure. FIG. 2 shows cooling device 100 with substrate 110, thermometer 120, cooler 130, and pouch 160.

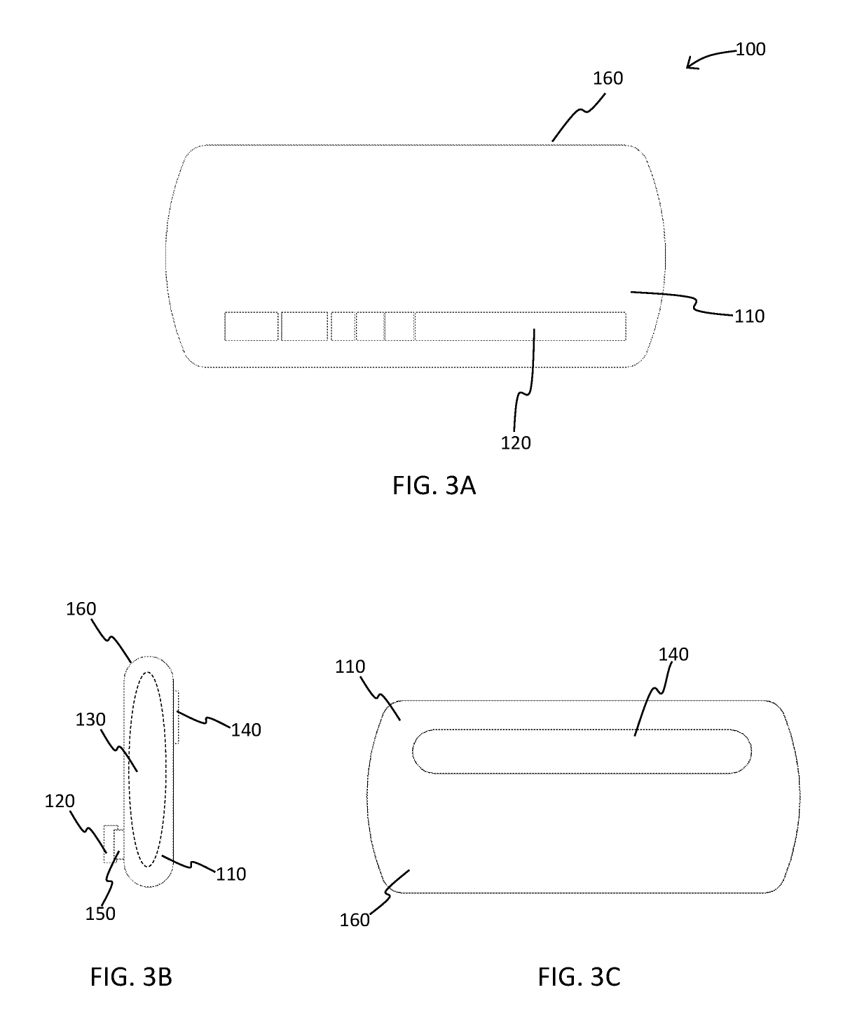

FIG. 3A shows a front view of cooling device 100 of FIG. 1, according to an embodiment of the present disclosure. As above, FIG. 3A illustrates cooling device 100, which can include substrate 110, thermometer 120, and pouch 160. FIG. 3B is a side view of cooling device 100 and depicts substrate 110, thermometer 120, cooler 130, connector 140, insulator 150, and pouch 160. Finally, FIG. 3C shows a back view of cooling device 100 and depicts substrate 110, connector 140, and pouch 160.

As shown, substrate 110 and cooler 130 are disposed inside of pouch 160. Thermometer 120 is disposed on insulator 150 that in turn is mounted to the surface of pouch 160. In some versions, connector 140 is disposed on the surface of pouch 160, as well. Substrate 110 serves to transfer heat from cooler 130 to pouch 160. In some versions, substrate 110 is water and in other versions, substrate 110 comprises a gelling agent. Connector 140 connects cooling device 100 to a portion of a patient's body to hold the cooling device against the patient's skin. Those of ordinary skill in the art will recognize diverse types of connector 140 to be useful in this invention. In some versions, connector 140 is a belt, a strap, an adhesive strip, or an arrangement of hook-and-loop fastener. Cooler 130 comprises a material that causes cooler 130 to cool when activated. For instance, in some versions, cooler 130 comprises water and an endothermic salt separated with an easily breakable barrier. When the barrier is broken, the water and salt combine and the combination absorbs energy from its surroundings, in this case substrate 110. Alternatively, substrate 110 comprises water and cooler 130 comprises an endothermic salt that is activated when cooler 130 is broken inside of substrate 110 distributing the endothermic salt to water similarly to the previous version. Using endothermic salts to provide cold therapy is well known to those of ordinary skill in the art. In some versions, the endothermic salt is selected from ammonium nitrate, urea, or ammonium chloride.

In some versions, substrate 110 is pre-cooled by placing it in a refrigerator or freezer. Sometimes this pre-cooling of cooling device 100 allows a version of cooling device 100 to dispense with cooler 130. Those of ordinary skill in the art will recognize that other ways of cooling substrate 110 exist. In various versions, these ways are useful in cooling device 100.

As shown in FIG. 3B, insulator 150 sits between pouch 160 and thermometer 120 so that thermometer 120 accurately reads the temperature of the patient's skin instead of the temperature of pouch 160 and substrate 110. Thermometer 120 can take many different forms as is known to those of ordinary skill in the art, which forms are useful in various versions of cooling device 100. For instance, thermometer 120 can be based on an electrical sensor or a color-changing plastic material or film.

In use, when a patient is outside or in a hot or heated environment, the thermometer indicates skin temperature when contacted with the skin. If the patient's skin temperature is too high, the patient or a caregiver activates the cooling device, in some versions by freeing the endothermic salt to dissolve in the water-based substrate. The cold cooling device 100 is placed against an appropriate region of the patient. In some versions, this region is the neck, armpits, or groin area. By doing this, cooling of the patient's body begins before other medical assistance arrives.

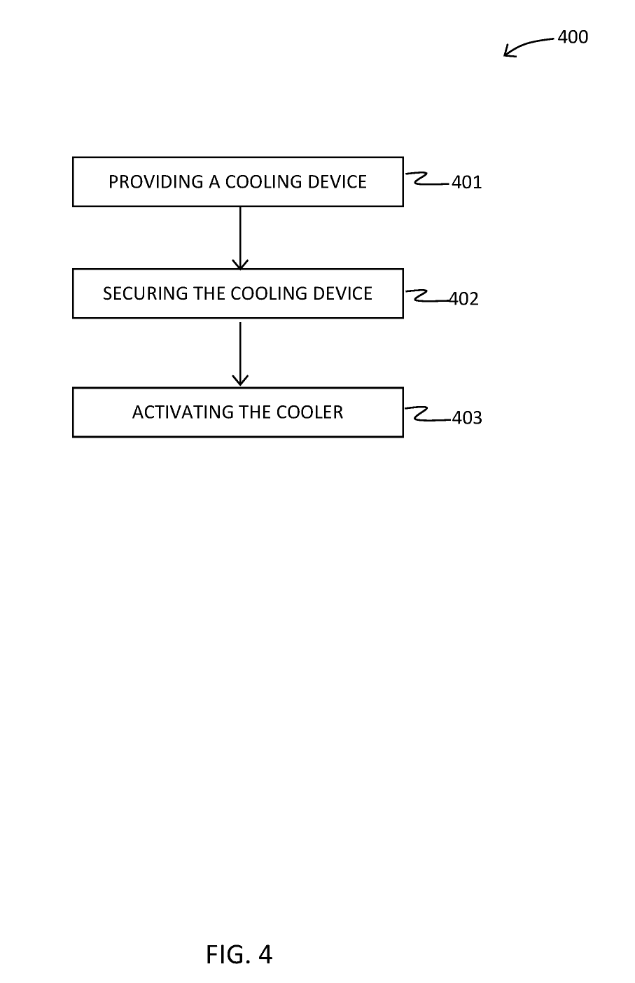

FIG. 4 is flow diagram 400 illustrating a method of using a cooling device 100, according to an embodiment of the present disclosure. As illustrated, the method of using a cooling device 100 may include the steps of: providing 401 a cooling device including a pouch; an activatable cooler disposed in the pouch; substrate disposed in the pouch; a thermometer affixed to the pouch; and a connector on an outer surface of the pouch; securing 402 the cooling device to a body region with the connector; and activating 403 the cooler.

The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for the cooling device 100 (e.g., different step orders within above-mentioned list, elimination or addition of certain steps, including or excluding certain maintenance steps, etc.), are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A cooling device comprising:
a pouch;
an activatable cooler disposed in the pouch;
substrate disposed in the pouch;
a thermometer affixed to the pouch;
and
a connector on an outer surface of the pouch wherein the connector secures the pouch to a body region.

2. The cooling device of claim 1, further comprising an insulator disposed between the thermometer and the pouch.

3. The cooling device of claim 2, wherein the insulator is an insulating strip.

4. The cooling device of claim 2, wherein the thermometer is electronic.

5. The cooling device of claim 4, wherein the thermometer comprises a plastic strip including color-changing material.

6. The cooling device of claim 5, wherein the connector comprises a belt and alternatively a strap.

7. The cooling device of claim 5, wherein the connector comprises adhesive disposed on the outer surface of the pouch.

8. The cooling device of claim 7, wherein the cooler comprises endothermic salt.

9. The cooling device of claim 8, wherein the pouch is a plastic bag and the endothermic salt is disposed in a sealed, frangible container.

10. The cooling device of claim 8, wherein the substrate includes a gel and alternatively water and a gelling agent.

11. A cooling device comprising:
a plastic bag;
an activatable cooling part including an endothermic salt in a sealed, frangible container disposed in the plastic bag;
substrate disposed in the pouch and including a gel;
a thermometer affixed to the pouch and comprising a plastic strip including color-changing;
an insulating strip disposed between the thermometer and the plastic bag;
and
adhesive material on an outer surface of the plastic bag wherein the adhesive material secures the plastic bag to a body region.
wherein the body region includes the neck, armpit, and groin regions.

12. A method of using a cooling device, the method comprising the steps of:
providing a cooling device including:
a pouch;
an activatable cooler disposed in the pouch;
substrate disposed in the pouch;
a thermometer affixed to the pouch;
and
a connector on an outer surface of the pouch wherein the connector is configured to secure the pouch to a body region;
securing the cooling device to a body region;
and
activating the cooler.
